(19) United States
(12) Patent Application Publication
(10) Pub. No.: US 2018/0356479 A1
(43) Pub. Date: Dec. 13, 2018

(54) Device for Reducing Magnetic Susceptibility Artifact

(71) Applicants: RIGSHOSPITALET, Copenhagen Ø (DK); UNIVERSITY OF COPENHAGEN, Copenhagen K (DK); REGION SJÆLLAND, Sorø (DK)

(72) Inventors: Anders OHLHUES, Frederiksberg C (DK); Carsten THOMSEN, Lyngby (DK); Mette MARKLUND, Hedehusene (DK); Michael Brun ANDERSEN, Ishøj (DK)

(21) Appl. No.: 15/775,544

(22) PCT Filed: Nov. 10, 2016
(86) PCT No.: PCT/DK2016/050362

§ 371 Date: May 11, 2018

(30) Foreign Application Priority Data
Nov. 12, 2015 (DK) PA 2015 70736

(51) Int. Cl. G01R 33/387; G01R 33/565; A61B 5/055; G01R 33/34

(52) U.S. Cl. CPC G01R 33/387; G01R 33/34; A61B 5/055; G01R 33/56536

(57) ABSTRACT

The present disclosure relates to a susceptibility artifact reducing vacuum bag for reducing local magnetic inhomogeneity inside a magnetic resonance imaging system, the vacuum bag comprising a mixture of diamagnetic composite material made of a diamagnetic material, such as pyrolytic graphite, and a filler material, the fraction of diamagnetic composite material and filler material selected such that the mixture has a net magnetic susceptibility corresponding substantially to the magnetic susceptibility of human tissue, the vacuum bag further comprising a valve for establishing an externally generated vacuum inside the vacuum bag.

The present disclosure relates to a device for reducing susceptibility artifacts in a magnetic resonance imaging system.

### BACKGROUND OF INVENTION

Magnetic resonance imaging (MRI) is a medical imaging technique used in radiology to investigate the anatomy and physiology of the body in both health and disease. MRI scanners use magnetic fields and radio waves to form images of the body. The technique is widely used in hospitals. The human body contains molecules having protons that become aligned in a magnetic field. An MRI scanner applies a strong magnetic field. The MRI scanner also produces a radio frequency (RF) current that creates a varying magnetic field. The protons absorb the varying field, which changes the spin of the protons. When the radio frequency current is turned off, the protons return to their original spin, which generates an RF signal that can be measured by receiving equipment and converted to an image.

Unfortunately, curves on the body of the patient create local inhomogeneity in the applied magnetic field. Optimal image quality of the MRI system requires a homogenous magnetic field. The introduction of a body in the magnetic field creates local magnetic inhomogeneity in the magnetic field. The inhomogeneity is caused by magnetic susceptibility differences between different material, typically between air, tissue, bone and fat. Susceptibility differences are particularly high between air and tissue, and therefore, the areas for example near the skin are often difficult to analyze by MRI scanning. Peripheral tumors located near the surfaces of the body are therefore sometimes problematic to analyze by MRI scanning. Magnetic susceptibility artifact refers to a distortion in the MR image, resulting from the described local magnetic field inhomogeneities.

There are techniques available for improving the homogeneity of the magnetic field in an MRI device. Shimming is one example of a technique for adjusting the homogeneity of a magnetic field. Shims with various shapes, including plates and coils, are used to adjust the magnetic field to compensate for local inhomogeneity. There are also a range of algorithms for actively using shimming techniques and for analyzing the received signals and for improving the quality of the obtained images.

### SUMMARY OF INVENTION

The present disclosure relates to a susceptibility artifact reducing vacuum bag for reducing local magnetic inhomogeneity inside a magnetic resonance imaging system, the vacuum bag comprising a mixture of diamagnetic composite material made of a diamagnetic material, such as pyrolytic graphite, and a filler material, wherein the fraction of diamagnetic composite material and filler material is selected such that the mixture has a net magnetic susceptibility corresponding substantially to the magnetic susceptibility of human tissue, the vacuum bag further comprising a valve for establishing an externally generated vacuum inside the vacuum bag. The magnetic resonance imaging system may be a combined resonance imaging system and positron emission tomography (PET) system. In such a system the MRI part may be used for soft tissue imaging and the PET part may be used for functional imaging.

Diamagnetic materials create an induced magnetic field in a direction opposite to an externally applied magnetic field, and are repelled by the applied magnetic field. In contrast, the opposite behavior is exhibited by paramagnetic materials. Diamagnetism is a quantum mechanical effect that occurs in all materials. Pyrolytic graphite is a very strong diamagnetic material. By mixing the diamagnetic material and the filler material such that the mixture has a net magnetic susceptibility corresponding substantially to the magnetic susceptibility of human tissue, and conforming the material to the body part to be examined, the image quality of an MRI system may improve significantly, in particular for areas close to the contour of the body of the patient. A mixture of approximately 8% pyrolytic graphite and 92% of a filler material having a low magnetic susceptibility has approximately the same diamagnetic equivalent as human tissue. The inventors have realized that by positioning such a material close to the skin of a patient undergoing an MRI scan, the magnetic inhomogeneity inside the MRI system is reduced and the image quality improves. Pyrolytic graphite is a good electrical conductor and therefore well suited for this purpose since, besides reducing the magnetic inhomogeneity, the emitted RF signals are not influenced considerably. The combination of the approximately same diamagnetic equivalent as human tissue, a relatively high electrical conductivity and a bag that positions the blend tightly to the skin without moving provides a very efficient device for reducing magnetic susceptibility artifacts in a magnetic resonance imaging system.

One challenge in achieving an improved image quality using the abovementioned mixture is often the positioning of the mixture very close to the tissue and maintaining it in a fixed position. Air gaps between the skin and the mixture may limit the potential improvements of the image quality and if the material is not maintained in a fixed position it may be difficult to use the potential of the concept. In the present disclosure, the mixture is provided in a susceptibility artifact reducing vacuum bag, typically a flexible bag such as a plastic bag. Preferably, the bag with content is formable after the body. Preferably, the bag is formable such that there is no, or at least a minimum gap, between the skin of the wearer and the gap, while the bag does not move. If the bag does not stay in a fixed position, local magnetic field will typically vary, which may have a negative impact on the image quality. By placing the bag on or around a body part in a non-vacuum configuration, i.e. when the vacuum bag is flexible and relatively loose in its shape, the bag can be adjusted such that it covers the body part. The body part may be for example the neck or one or two breasts of a body. Vacuum is then applied, which causes the volume of the vacuum bag to shrink and conform tightly to the body part. When vacuum is applied, a wearer may feel that that the bag is tightened around a body part in the sense that it closes the gaps between the skin and the bag and stays in a fixed position. Typically, the vacuum bag with content (mixture) becomes significantly stiffer in the vacuum configuration. When vacuum is applied, the bag conforms after the body contour and ensures that substantially no air gaps between the skin and the bag is present. When the MRI session is finished, the valve of the vacuum bag can be opened, which causes air to flow into the vacuum bag, which regains its flexibility and can be removed easily from the body part. The vacuum bag is then ready to be reused.

In a preferred embodiment, the mixture of diamagnetic composite material and filler material form a granular material. The diamagnetic composite material and filler material may for example be obtained by melting the materials, mixing them into a homogenous substance and forming beads and/or grains of the substance. In relation to the presently disclosed susceptibility artifact reducing vacuum bag, it may be beneficial to use a filler material that renders the beads and/or grains elastic and/or deformable, possibly a rubber or rubber-like material. When vacuum is established in the vacuum bag, the elastic beads/grains may serve several purposes. The elasticity enables a high degree of conformity of the vacuum bag on the body portion. Furthermore, since one aspect of the invention is to reduce the magnetic inhomogeneity, it is advantageous if the mixture of materials is as precise as possible.

If inflexible grains/beads were to be used inside the vacuum bag, there would be small gaps between the grains/beads even when vacuum has been established. If the grains/beads are instead elastic, the grains/beads will be deformed and more closely packed in the vacuum configuration.

Suppression of fat signal is used in MRI images when the fat signal causes artifacts or otherwise obscures a tissue of interest. There are a number of fat suppression methods. Fat saturation refers to a technique that selectively saturates fat protons prior to acquiring data. The technique requires a very homogeneous magnetic field. The presently disclosed susceptibility artifact reducing vacuum bag may reduce local inhomogeneities in an MRI system significantly.

The field strength of the magnet in an MRI system is measured in teslas. Many systems operate at 1.5 T. However, commercial systems are available between 0.2 T-7 T. The presently disclosed susceptibility artifact reducing vacuum bag may be used in any MRI system.

These and other aspects of the invention are set forth in the following detailed description of the invention.

### DESCRIPTION OF DRAWINGS

FIG. 1 shows an embodiment of the presently disclosed susceptibility artifact reducing vacuum bag with a manual handheld vacuum pump and a tube to be connected to the valve of the vacuum bag.

FIG. 2 shows the susceptibility artifact reducing vacuum bag; manual handheld vacuum pump; and tube of FIG. 1 in a connected configuration.

FIG. 3 shows a portion of the susceptibility artifact reducing vacuum bag of FIG. 1 and FIG. 2 from a different angle.

FIG. 4 shows an embodiment of the presently disclosed the susceptibility artifact reducing vacuum bag placed around the neck of a user. The bag is in a vacuum configuration and conforms tightly to the neck.

FIG. 5 shows a pair of MRI images of the neck region of a patient, of which FIG. 5A is the result without the presently disclosed susceptibility artifact reducing vacuum bag, and FIG. 5B is the result when using an embodiment of the presently disclosed susceptibility artifact reducing vacuum bag.

FIG. 6 shows a pair of MRI images of the neck region of a patient, of which FIG. 6A is the result without the presently disclosed susceptibility artifact reducing vacuum bag, and FIG. 6B is the result when using an embodiment of the presently disclosed susceptibility artifact reducing vacuum bag.

### DETAILED DESCRIPTION OF THE INVENTION

The present disclosure relates to a susceptibility artifact reducing vacuum bag for reducing local magnetic inhomogeneity inside a magnetic resonance imaging system, the vacuum bag comprising a mixture of diamagnetic composite material made of a diamagnetic material, such as pyrolytic graphite, and a filler material, wherein the fraction of diamagnetic composite material and filler material is selected such that the mixture has a net magnetic susceptibility corresponding substantially to the magnetic susceptibility of human tissue, the vacuum bag further comprising a valve for establishing an externally generated vacuum inside the vacuum bag.

As stated, by mixing the diamagnetic material and the filler material such that the mixture has a net magnetic susceptibility corresponding substantially to the magnetic susceptibility of human tissue, and conforming the material to the body part to be examined, the image quality may improve significantly. Magnetic susceptibility is a quantitative measure of the extent to which a material may be magnetized in relation to a given applied magnetic field. A mixture of approximately 8% pyrolytic graphite and 92% of a filler material having a low magnetic susceptibility (approximately zero) has approximately the same diamagnetic equivalent as human tissue. Diamagnetism is a property of all materials and makes a contribution to the material's response to a magnetic field. Other forms of magnetism are ferromagnetism and paramagnetism. Substances that mostly display diamagnetic behavior are termed diamagnetic materials, or diamagnets. Diamagnetic materials include water, wood, most organic compounds such as petroleum and some plastics, and many metals including copper, particularly the heavy ones with many core electrons, such as mercury, gold and bismuth. Diamagnetic materials have a relative magnetic permeability that is less than or equal to 1, and therefore a magnetic susceptibility less than or equal to 0, since susceptibility is defined as χᵥ=μᵥ−1. Diamagnetic materials have a characteristic negative magnetic susceptibility. The magnetic susceptibility of water is χᵥ=−9×10⁻⁶. The magnetic susceptibility of bismuth is χᵥ=−1.5×10⁻⁴. The magnetic susceptibility of pyrolytic graphite is χᵥ=−4.5×10⁻⁴. Superconductors may be considered perfect diamagnets (χᵥ=−1). The inventors have realized that by positioning such a material close to the skin of a patient undergoing an MRI scan, the magnetic inhomogeneity inside the MRI system is reduced and the image quality improves. Pyrolytic graphite is also a good electrical conductor and therefore well suited for this purpose since, besides reducing the magnetic inhomogeneity, the emitted RF signals are not influenced considerably.

As stated, one challenge in achieving an improved image quality in MRI systems using the abovementioned mixture is often the positioning of the mixture very close to the tissue and maintaining it in a fixed position. In the present disclosure, the mixture is provided in a susceptibility artifact reducing vacuum bag, typically a flexible bag such as a plastic bag. By placing the bag on or around a body part in a non-vacuum configuration, i.e. when the vacuum bag is flexible and relatively loose in its shape, the bag can be adjusted such that it covers the body part. The vacuum bag may be configured and/or shaped for any body part that could benefit from a reduced magnetic inhomogeneity in an MRI system. The body part may be for example the neck or one or two breasts of a body. The body part referred to may also be the foot, or leg, or knee, or arm, or hand, or shoulder, or back, or head of a person. Vacuum is then applied, which causes the vacuum bag to shrink and conform tightly to the body part. Typically, the vacuum bag with content (mixture) becomes significantly stiffer. When vacuum is applied, the bag conforms after the body contour and ensures that substantially no air gaps between the skin and the bag is present.

Vacuum in the present disclosure shall not be construed as absolute vacuum but a decreased pressure inside the vacuum bag that allows the bag with content to take the needed properties. Generally “vacuum” may be defined as a region with a gaseous pressure less than the ambient pressure, i.e. the surrounding atmospheric pressure. At sea level on Earth the atmospheric pressure is approximately 1 bar, i.e. 1000 mbar at 25° C. The present disclosure refers to a “vacuum bag” with the meaning that by connecting its valve to a pump, the pressure inside the bag can be reduced such that the mixture inside fills “all” the interior of the bag and the bag takes a more rigid character. The person skilled in the art would be capable of construing the required level of negative pressure to use the presently disclosed susceptibility artifact reducing vacuum bag for reducing local magnetic inhomogeneity inside a magnetic resonance imaging system.

“Low”, “medium” and “high” vacuum at sea level on earth in mbar may also be defined according to the following table.

| | pressure (mbar) |
|---|---|
| Atmospheric pressure | 1000 |
| Low vacuum | 1000 to 100 |
| Medium vacuum | 100 to 0.001 |
| High vacuum | <0.001 |

### Mixture Embodiments

In one embodiment of the presently disclosed susceptibility artifact reducing vacuum bag, the mixture of diamagnetic composite material and filler material form a granular material. A granular material is a conglomeration of discrete solid, macroscopic particles characterized by a loss of energy whenever the particles interact. Examples of granular materials are snow, nuts, coal, sand, rice, coffee, corn flakes, fertilizer and ball bearings. Powders are a special class of granular material due to their small particle size. A powder is a material composed of very fine particles that are not cemented together. The mixture of diamagnetic composite material made of a diamagnetic material and a filler material of the bag of the present disclosure may be two powders. In one embodiment the diamagnetic composite material made of a diamagnetic material may be pyrolytic graphite powder. The particles of the pyrolytic graphite powder may have a diameter of less than 3 mm, preferably less than 2 mm, more preferably less than 1 mm, even more preferably less than 100 μm, most preferably less than 20 μm. The filler material may comprise organic material, such as flour, in the form of a powder. It may also comprise non-organic, or a mix of organic and non-organic material. The particles of the filler powder may have a diameter of less than 3 mm, preferably less than 2 mm, more preferably less than 1 mm, even more preferably less than 100 μm, most preferably less than 20 μm. Preferably, the concentrations of diamagnetic composite material made of a diamagnetic material and filler material are evenly distributed in the bag. In the presently disclosed susceptibility artifact reducing vacuum bag, the net magnetic susceptibility may be achieved by mixing particles, e.g. grains or beads of the diamagnetic composite material and other grains or beads of the filler material. However, in one embodiment, the mixing takes place already in the manufacturing of a mixed material. The mixture is thereby evenly distributed in the mixed material, which may then be divided into beads and/or grains. In contrast to the mixing of beads/grains from two different materials, the composite material and filler material is evenly distributed already at the bead/grain level and there is therefore no risk that areas with a higher or lower concentration of one of the materials are created in the vacuum bag. The mixture of diamagnetic composite material and filler material, wherein the materials are evenly distributed in a material forming beads and/or grains can be said to ensure a homogenous magnetic susceptibility in the vacuum bag.

The beads and/or grains may take any suitable shape. In one embodiment, the beads and/or grains are cylindrical, or spherical, or conical, or cubical, or rectangular, or pyramid shaped, or combinations thereof. Also the size of the beads/and or grains may take any suitable size. In one embodiment, the size of the beads and/or grains is less than 10 mm, or less than 9 mm, or less than 8 mm, or less than 7 mm, or less than 6 mm, or less than 5 mm, or less than 4 mm, or less than 3 mm, or less than 2 mm, or less than 1 mm, or less than 0.5 mm, or less than 0.3 mm, or less than 0.1 mm. The size in this regard shall be construed as the diameter of a part of the grains/beads. Preferably, the size is selected such that, combined with the grains/beads being elastic, it is possible to substantially eliminate the gaps between the grains/beads in the vacuum bag by establishing vacuum inside the bag, thereby deforming the grains/beads. Therefore, in one embodiment of the presently disclosed susceptibility artifact reducing vacuum bag, the beads and/or grains are elastic and/or deformable by establishment of vacuum in the susceptibility artifact reducing vacuum bag. The deformation and possible configurations of the presently disclosed vacuum bag is presented in further detail below.

Configurations (Non-Vacuum Configuration, Vacuum Configuration)

In one embodiment, the susceptibility artifact reducing vacuum bag is, in a non-vacuum configuration, configured to be positioned on a portion of a human body, and, in a vacuum configuration, configured to conform tightly on said portion, the vacuum configuration corresponding to a state wherein vacuum has been established. In the non-vacuum configuration, the vacuum bag is flexible, which makes it possible to place said vacuum bag near or in direct connection with the body portion to be examined in the MRI session. In this configuration, the vacuum bag can be compared to a bean bag used as seating furniture, in terms of shape and flexibility. Such a bag is typically easily placed on a body portion, but has a tendency to move if the user moves. Furthermore, there may be a gap of air between the bag and the body part and there may be gap between the beans (or any grain or beads in the bag). Therefore, in the vacuum configuration, wherein vacuum has been established in the bag, the content of the bag is compressed and the shape of the bag takes the shape of the contour of the body part. FIG. 4 shows an example of the presently disclosed susceptibility artifact reducing vacuum bag in a vacuum configuration, wherein the vacuum bag is placed around the neck of a patient. It can be noted that the established vacuum retains the shape of the susceptibility artifact reducing vacuum bag in this configuration. In this example the vacuum bag is in a tight position against the skin of the wearer and does not move even if the wearer tries to move the neck. In the example, the vacuum bag has typically previously been positioned while the bag was in a non-vacuum configuration and preferably relatively flexible. In the vacuum position shown in FIG. 4, the vacuum bag is substantially rigid and stiff and does not move in relation to the patient. In one embodiment, the susceptibility artifact reducing vacuum bag and the mixture of diamagnetic composite material form a substantially stiff and/or rigid assembly in the vacuum configuration. In a specific embodiment, the neck region is substantially locked in relation to the presently disclosed vacuum bag in the vacuum configuration. In one embodiment, the susceptibility artifact reducing vacuum bag has a shape preventing it from moving or falling off from the position on a human body in the vacuum configuration and in one embodiment, the susceptibility artifact reducing vacuum bag is configured to conform to the neck of a human body.

Furthermore, in the vacuum configuration, the beads and/or grains being are packed closely in the vacuum, thereby increasing the density of the susceptibility artifact reducing vacuum bag in the vacuum configuration. As a consequence, the space between the beads and/or grains is reduced, which makes it easier to calculate the net magnetic susceptibility of the vacuum bag including its content. If the beads and/or grains are elastic, as described above, the beads and/or grains may be deformed in the vacuum configuration, thereby eliminating the gaps between the beads/grains in the vacuum configuration. This has the further advantage that the total magnetic susceptibility of the mixture can be adjusted by increasing or decreasing the level of vacuum to find the optimal level both in terms of position of the bag on the body portion and in terms of level of net magnetic susceptibility taking into account small air gaps between the beads/grains. The elasticity of the mixture or beads/grains is further described below.

The valve for establishing an externally generated vacuum inside the vacuum bag may be a relatively simple design through which the bag can be connected to a vacuum pump configured to generate vacuum inside the vacuum bag. For those skilled in art it would be possible to design the connection between the vacuum bag and the vacuum pump in several ways. In one embodiment, the valve has a first configuration in which air can only flow from the inside of the susceptibility artifact reducing vacuum bag, and a second configuration in which air can flow both in and out to/from the susceptibility artifact reducing vacuum bag. The first configuration typically corresponds to a configuration in which vacuum is generated and/or maintained, while the second configuration corresponds to the non-vacuum configuring of the vacuum bag or the process of exiting the vacuum state of the bag after an MRI session. The re-entry of air into the vacuum bag may be blocked in the first configuration. There may also be a third configuration of the valve, in which the valve is completed locked for air flowing in and out to/from the susceptibility artifact reducing vacuum bag. This can be seen as the valve simply being plugged or sealed in the third configuration.

Sizes and Shapes

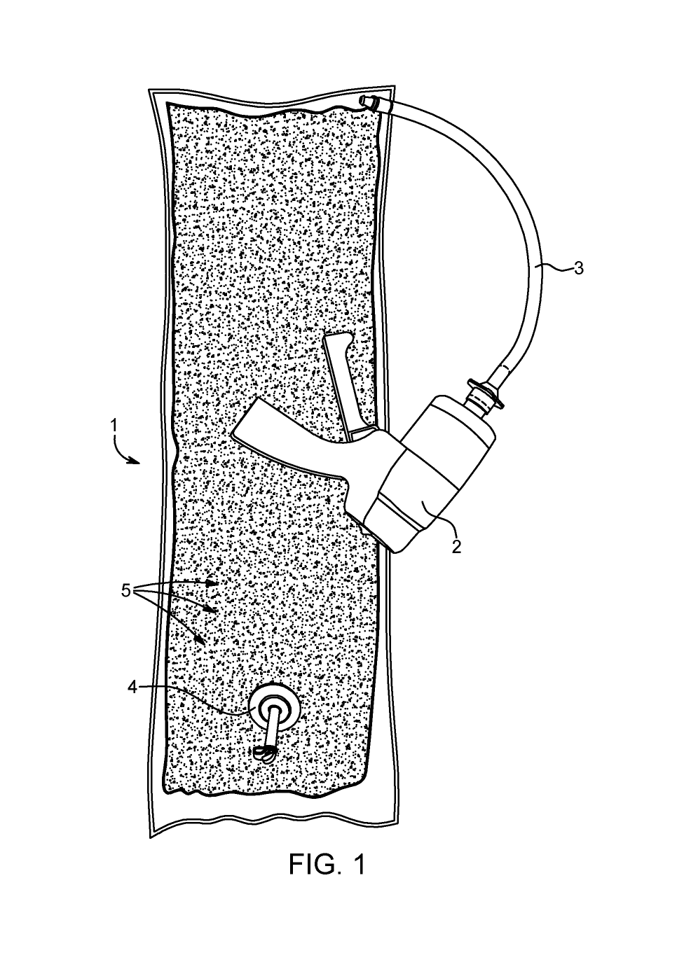

The susceptibility artifact reducing vacuum bag may be provided in a range of sizes and shapes in order to be used with different body portions. In one embodiment, the susceptibility artifact reducing vacuum bag is substantially rectangular (as shown in e.g. FIG. 1) having a width less than 400 mm, or less than 350 mm, or less than 300 mm, or less than 250 mm, or less than 200 mm, or less than 150 mm, or less than 100 mm. In one embodiment, the length of the susceptibility artifact reducing vacuum bag is less than 1000 mm, or less than 900 mm, or less than 800 mm, or less than 700 mm, or less than 600 mm, or less than 500 mm, or less than 400 mm. As stated, in one embodiment, the susceptibility artifact reducing vacuum bag is configured to conform to the neck of a human body. The vacuum bag may have a height of less than 70 mm, such as less than 50 mm, preferably less than 30 mm, more preferably less than 20 mm, even more preferably less than 10 mm.

In a further embodiment, the susceptibility artifact reducing vacuum bag is configured to conform to at least one breast of a human body. A conventional MRI unit is shaped as a large cylinder into which the patient is introduced. Typically the patient lies on a movable examination table that slides into the bore of the MRI system. For a breast MRI, the patient usually lies face down, with the breasts positioned through openings in the table. Typically the breasts hang through the openings in a recess. Therefore, in one embodiment, the presently disclosed susceptibility artifact reducing vacuum bag is configured to be placed in recess, preferably a cushioned recess, wherein a breast is positioned in the recess. Furthermore, in one embodiment, the presently disclosed susceptibility artifact reducing vacuum bag is substantially cup-shaped or substantially conical or substantially hemispherical. As for the embodiment suitable for the neck of a patient, in the embodiment suitable for at least one breast, the vacuum bag is configured to conform tightly and not move in relation to the breast in the vacuum configuration. For the embodiment in which the susceptibility artifact reducing vacuum bag is configured to conform to at least one breast of a human body, the fact that the vacuum bag is adjustable by increasing or decreasing the level of vacuum may be a further advantage. Since compressing the breast may reduce the blood flow to e.g. a tumor to be scanned, which is not a desirable situation, the fact that the vacuum bag can be vacuum adjusted to a level that it conforms to the breast but does not compress the breast more than necessary. Hence, the design of the presently disclosed susceptibility artifact reducing vacuum bag may enable useful trade-off functionality.

Materials

As stated, there are a number of diamagnetic materials. Pyrolytic graphite is an unusually strong diamagnetic material. It is also a good electrical conductor and therefore well suited for this purpose since, besides reducing the magnetic inhomogeneity, the emitted RF signals are not influenced considerably. In one embodiment, the diamagnetic material of the presently disclosed vacuum bag has an electrical conductivity higher than 10⁵ S/m, or higher than 10⁶ S/m, or higher than 10⁷ S/m, or higher than 10⁸ S/m. Furthermore, pyrolytic graphite does not heat considerably inside an MRI scanner. For these reasons, in a preferred embodiment, the diamagnetic material inside the presently disclosed susceptibility artifact reducing vacuum bag is pyrolytic graphite.

The filler material of the mixture in the susceptibility artifact reducing vacuum bag may be selected from a number of polymers. The polymer may be selected from a group of synthetic plastics, for example within the groups thermoplastics, thermosets, elastomers and synthetic fibers, such as Polystyrene, Low Density Polyethylene, High Density Polyethylene, Polypropylene (PP), Polyvinyl Chloride (PVC), Polystyrene (PS), Thermoplastic polyurethanes, and have different tensile strengths and elasticity. The filler material may also be selected from the group of silicone polymers, epoxy polymers, acrylate polymers, or elastic polymers. In the present invention, elastic refers to the ability to resist a distorting influence or stress and to return to its original size and shape when the stress is removed. If the material is elastic, the object will return to its initial shape and size when the stress/force is removed. An elastomer is a polymer with viscoelasticity (having both viscosity and elasticity) and very weak inter-molecular forces, generally having low Young's modulus and high failure strain compared with other materials. The term, which is derived from elastic polymer, is often used interchangeably with the term rubber, although the latter is preferred when referring to vulcanisates. In one embodiment, the filler material is elastic. The filler material in the presently disclosed susceptibility artifact reducing vacuum bag may be selected from the group of unsaturated rubbers, such as Natural polyisoprene (natural rubber), Synthetic polyisoprene (isoprene rubber), Polybutadiene (butadiene rubber), Chloroprene rubber (CR), Butyl rubber, Halogenated butyl rubbers, Styrene-butadiene Rubber, Nitrile rubber, Hydrogenated Nitrile Rubbers, and/or from saturated rubbers, such as Ethylene Propylene rubber (EPR) and EPDM rubber (ethylene propylene diene rubber), Epichlorohydrin rubber (ECO), Polyacrylic rubber (ACM, ABR), Silicone rubber (SI, Q, VMQ), Fluorosilicone Rubber (FVMQ), Fluoroelastomers (FKM, and FEPM), Perfluoroelastomers (FFKM), Polyether block amides (PEBA), Chlorosulfonated polyethylene (CSM), Ethylene-vinyl acetate (EVA).

The filler material preferably has a low magnetic susceptibility. The filler material is preferably also be substantially non-conducting or has low conductivity. In one embodiment, small size particles form the filler material. The filler material may also comprise organic material, such as flour, wood, sugar, and grain or any material having a low magnetic susceptibility.

The vacuum bag may be made of any suitable air-tight material, preferably made of plastic material, such as polyethylene, or polyvinyl chloride (PVC), or silicone plastic.

As stated, the fraction of diamagnetic composite material and filler material may be selected such that the mixture has a net magnetic susceptibility corresponding substantially to the magnetic susceptibility of human tissue. A mixture of approximately 8% pyrolytic graphite and 92% of a filler material having a low magnetic susceptibility has approximately the same diamagnetic equivalent as human tissue. In one embodiment, the mixture comprises from about 4% v/v to about 12% v/v of the diamagnetic material, or about 5% v/v to about 11% v/v of the diamagnetic material, or about 6% v/v to about 10% v/v of the diamagnetic material, or about 7% v/v to about 9% v/v of the diamagnetic material, or about 7.5% v/v to about 8.5% v/v of the diamagnetic material, or about 7.8% v/v to about 8.2% v/v of the diamagnetic material, or about 7.9% v/v to about 8.1% v/v of the diamagnetic material, such as about 8.0% v/v, or about 8.1% v/v, or about 8.2% v/v, or about 8.3% v/v, or about 8.4% v/v, or about 8.5% v/v, or about 7.9% v/v, or about 7.8% v/v, or about 7.7% v/v, or about 7.6% v/v, or about 7.5% v/v of the diamagnetic material. The volume percentage shall be construed such that only the volume is expressed in diamagnetic material and filler material only—possible gaps between grains or beads are not to be counted in the above fractions.

Application

The present disclosure further relates to a method for applying the susceptibility artifact reducing vacuum bag for reducing local magnetic inhomogeneity. The method may be used to reduce local magnetic inhomogeneity of a portion of a body inside a magnetic resonance imaging system. In one embodiment the method for reducing local magnetic inhomogeneity of a portion of a body inside a magnetic resonance imaging system comprises the steps of:
providing a susceptibility artifact reducing vacuum bag comprising a mixture of diamagnetic composite material made of a diamagnetic material and a filler material, the fraction of diamagnetic composite material and filler material selected such that the mixture has a net magnetic susceptibility corresponding substantially to the magnetic susceptibility of human tissue;
positioning the bag on and/or around the portion of the body, wherein the vacuum bag is in a non-vacuum configuration;
establishing a vacuum in the vacuum bag, thereby entering a vacuum configuration of the vacuum bag, wherein the vacuum bag conforms tightly to the body.

In one example an oblong or elongated version of the vacuum bag, as shown in FIG. 4, is placed around the neck of the user. The bag is placed such that it covers areas that are known to be inhomogeneous in MRI imaging. Preferably, the vacuum bag is loose and flexible in this non-vacuum configuration. In a second step vacuum is established through a valve. Typically, the vacuum is established by means of a pump connected to the valve. During this step the user will typically that as the vacuum bag becomes stiffer and conforms more and more tightly to the neck. When the vacuum configuration has been reached, the vacuum bag is substantially stiff and does not easily change shape. Preferably the valve is closed after reaching the vacuum configuration. The bag then stays in this configuration during the MRI session. After the session, the valve can be opened again and the vacuum bag regains its flexibility and can be removed again and possibly used by another user.

Vacuum bags according to the present disclosure may be designed for any suitable body part.

### DETAILED DESCRIPTION OF DRAWINGS

The invention will in the following be described in greater detail with reference to the accompanying drawings. The drawings are exemplary and are intended to illustrate some of the features of the presently disclosed susceptibility artifact reducing vacuum bag, and are not to be construed as limiting to the presently disclosed invention.

FIG. 1 shows an embodiment of the presently disclosed susceptibility artifact reducing vacuum bag 1 with a manual handheld vacuum pump 2 and a tube 3 to be connected to the valve 4 of the vacuum bag. The susceptibility artifact reducing vacuum bag 1 of this example is filled with a large number of elastic beads 5.

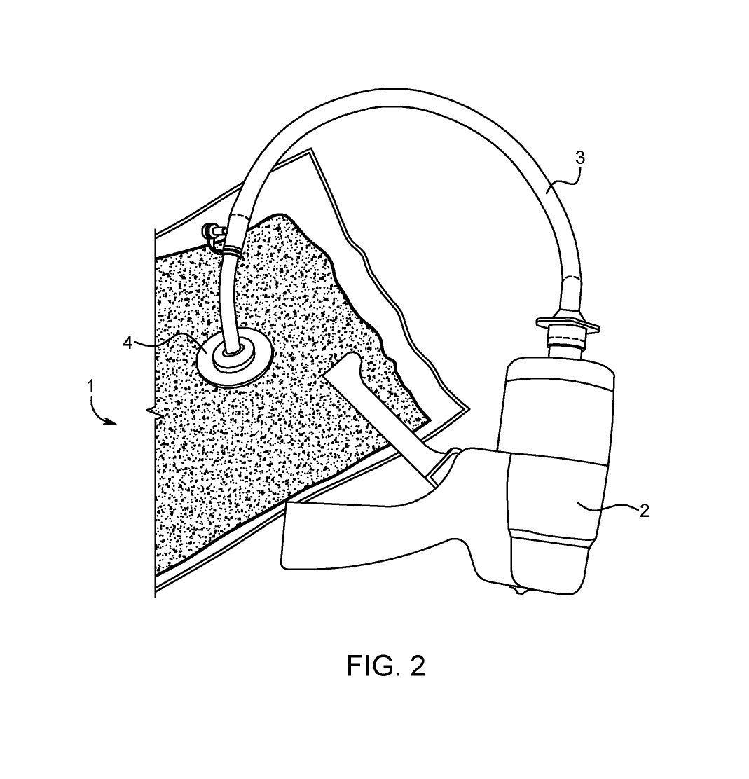

FIG. 2 shows the susceptibility artifact reducing vacuum bag 1; manual handheld vacuum pump 2; tube 3; and valve 4 of FIG. 1 in a connected configuration.

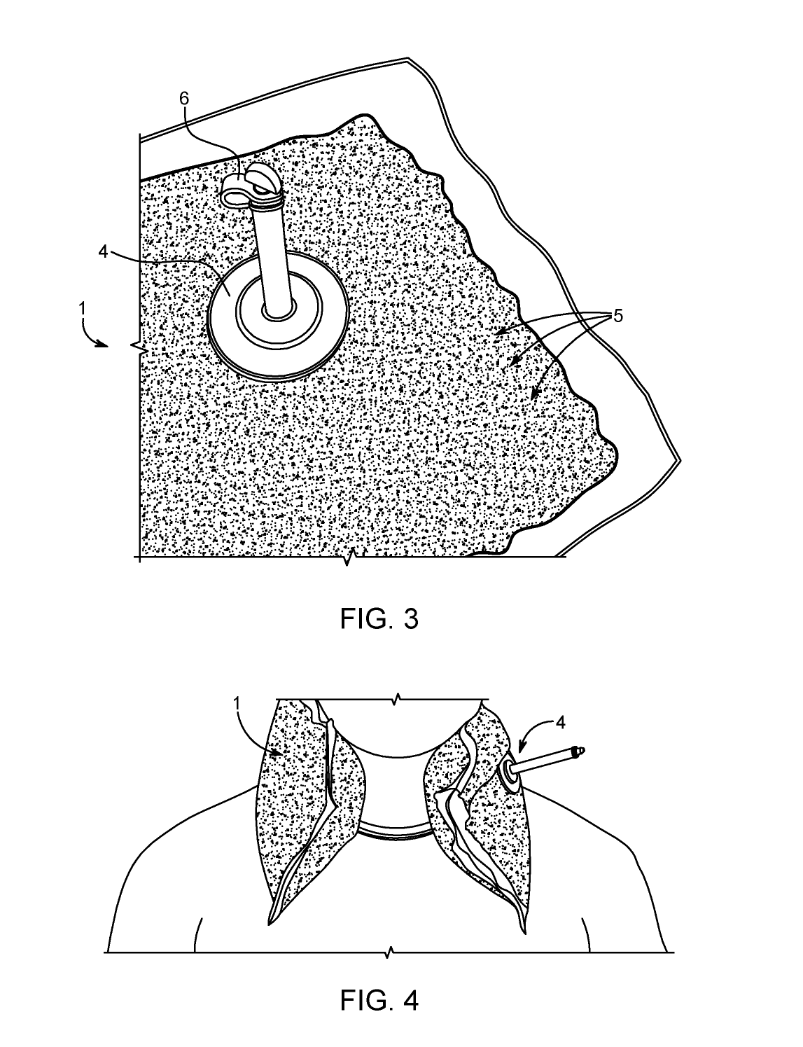

FIG. 3 shows a portion of the susceptibility artifact reducing vacuum bag of FIG. 1 and FIG. 2 from a different angle. The susceptibility artifact reducing vacuum bag 1 of this example is filled with a large number of elastic beads 5. In the example the elastic beads 5 are cylindrical in their shape.

FIG. 4 shows an embodiment of the presently disclosed the susceptibility artifact reducing vacuum bag 1 placed around the neck of a user. The bag is in a vacuum configuration and conforms tightly to the neck. In this configuration, the vacuum bag 1 is substantially rigid and stiff and does not move in relation to the patient.

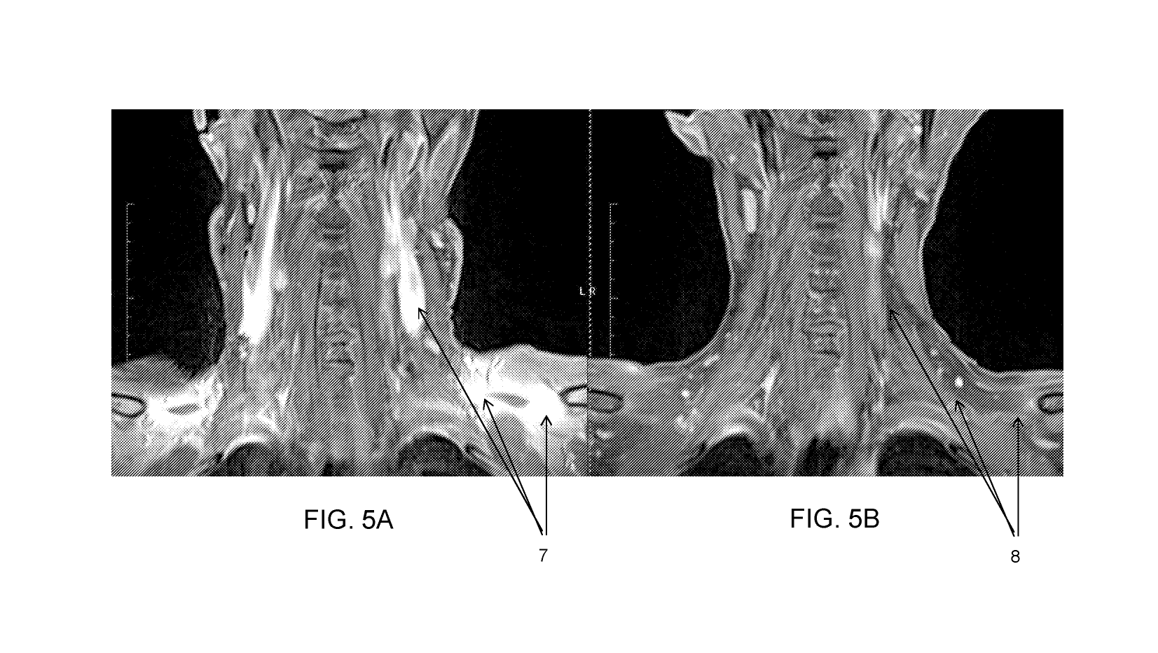

FIG. 5 shows a pair of MRI images with the quality of a 1.5 T MRI scanner of the neck region of a patient, of which FIG. 5A is the result without the presently disclosed susceptibility artifact reducing vacuum bag, and FIG. 5B is the result when using an embodiment of the presently disclosed susceptibility artifact reducing vacuum bag. The image in FIG. 5A has a suboptimal fat suppression due to local inhomogeneity in the applied magnetic field. The areas having suboptimal fat suppression are indicated 7 in the figure. FIG. 5B has significantly better fat suppression (corresponding areas 8) as a result of using an embodiment of the presently disclosed susceptibility artifact reducing vacuum bag.

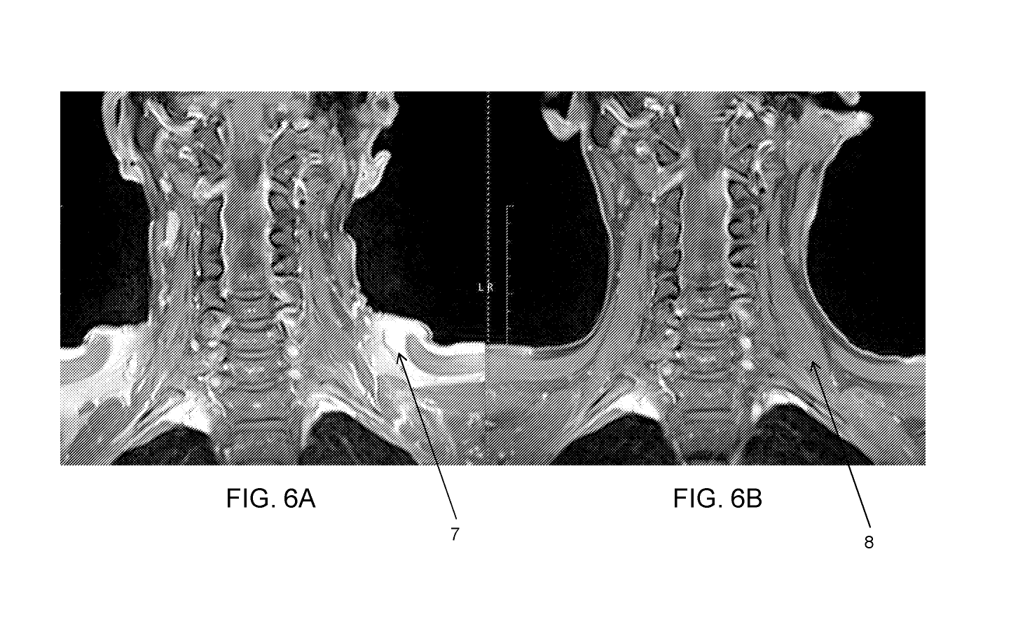

FIG. 6 shows a pair of MRI images with the quality of a 1.5 T MRI scanner of the neck region of a patient, of which FIG. 6A is the result without the presently disclosed susceptibility artifact reducing vacuum bag, and FIG. 6B is the result when using an embodiment of the presently disclosed susceptibility artifact reducing vacuum bag. The image in FIG. 6A has a suboptimal fat suppression due to local inhomogeneity in the applied magnetic field. The areas having suboptimal fat suppression are indicated 7 in the figure. FIG. 6B has significantly better fat suppression (corresponding areas 8) as a result of using an embodiment of the presently disclosed susceptibility artifact reducing vacuum bag.

### Examples

Experiments have been conducted to test the performance of the device. One experiment involves manual inspection of MR images and a comparison of images when susceptibility artifact reducing vacuum bag was not used and corresponding images when the susceptibility artifact reducing vacuum bag was positioned at the body part and a vacuum was established such that the vacuum bag conformed tightly to the body part. The coronal T1 SE fat saturation and axial EPI diffusion were compared for 10 test patients. It was found that all 10 images obtained using the susceptibility artifact reducing vacuum bag improved the image quality in terms of T1 SE fat saturation and axial EPI diffusion.

| | Coronal T1 SE | Axial EPI |
|---|---|---|
| With bag vs. without bag | fat saturation | diffusion |
| No change or worse | 0 | 0 |
| Improvement | 10 | 10 |

1. A susceptibility artifact reducing vacuum bag for reducing local magnetic inhomogeneity inside a magnetic resonance imaging system, the vacuum bag comprising a mixture of diamagnetic composite material made of a diamagnetic material, such as pyrolytic graphite, and a filler material, the fraction of diamagnetic composite material and filler material selected such that the mixture has a net magnetic susceptibility corresponding substantially to the magnetic susceptibility of human tissue, the vacuum bag further comprising a valve for establishing an externally generated vacuum inside the vacuum bag.

2. The vacuum bag according to any of the preceding claims, wherein the diamagnetic material has an electrical conductivity higher than 10⁵S/m, or higher than 10⁶ S/m, or higher than 10⁷S/m, or higher than 10⁸ S/m.

3. The vacuum bag according to any of the preceding claims, wherein the diamagnetic composite material made of a diamagnetic material is a first powder, such as a pyrolytic graphite powder.

4. The vacuum bag according to any of the preceding claims, wherein the filler material is a second powder, such as flour.

5. The vacuum bag according to any of the preceding claims, wherein the first and/or second powder(s) have a diameter of less than 3 mm, preferably less than 2 mm, more preferably less than 1 mm, even more preferably less than 100 μm, most preferably less than 20 μm.

6. The vacuum bag according to any of the preceding claims, wherein concentrations of the diamagnetic composite material and filler material are distributed evenly in the vacuum bag.

7. The vacuum bag according to any of the preceding claims, the mixture of diamagnetic composite material and filler material forming a granular material.

8. The susceptibility artifact reducing vacuum bag according to any of the preceding claims, wherein the mixture of diamagnetic composite material and filler material is evenly distributed in a material forming beads and/or grains.

9. The susceptibility artifact reducing vacuum bag according to claim 8, wherein the beads and/or grains are cylindrical, or spherical, or conical, or cubical, or rectangular, or pyramid shaped, or combinations thereof.

10. The susceptibility artifact reducing vacuum bag according to any of claims 8-9, wherein the size of the beads and/or grains is less than 10 mm, or less than 9 mm, or less than 8 mm, or less than 7 mm, or less than 6 mm, or less than 5 mm, or less than 4 mm, or less than 3 mm, or less than 2 mm, or less than 1 mm, or less than 0.5 mm, or less than 0.3 mm, or less than 0.1 mm.

11. The susceptibility artifact reducing vacuum bag according to any of claims 8-10, wherein the beads and/or grains are elastic and/or deformable by establishment of vacuum in the susceptibility artifact reducing vacuum bag.

12. The susceptibility artifact reducing vacuum bag according to any of the preceding claims, wherein the susceptibility artifact reducing vacuum bag in a non-vacuum configuration is configured to be positioned on a portion of a human body, and in a vacuum configuration is configured to conform tightly on said portion, the vacuum configuration corresponding to a state wherein vacuum has been established.

13. The susceptibility artifact reducing vacuum bag according to claim 12, the established vacuum retaining the shape of the susceptibility artifact reducing vacuum bag in the vacuum configuration.

14. The susceptibility artifact reducing vacuum bag according to any of claims 12-13, the susceptibility artifact reducing vacuum bag and the mixture of diamagnetic composite material forming a substantially stiff and/or rigid assembly in the vacuum configuration.

15. The susceptibility artifact reducing vacuum bag according to any of claims 12-14, the beads and/or grains being packed closely in the vacuum configuration, thereby increasing the density of the susceptibility artifact reducing vacuum bag in the vacuum configuration.

16. The susceptibility artifact reducing vacuum bag according to any of claims 12-15, the elastic beads and/or grains being deformed in the vacuum configuration.

17. The susceptibility artifact reducing vacuum bag according to any of claims 12-16, the susceptibility artifact reducing vacuum bag having a shape preventing it from moving or falling off from the position on a human body in the vacuum configuration.

18. The susceptibility artifact reducing vacuum bag according to any of the preceding claims, wherein the valve has a first configuration in which air can only flow from the inside of the susceptibility artifact reducing vacuum bag, and a second configuration in which air can flow both in and out to/from the susceptibility artifact reducing vacuum bag.

19. The susceptibility artifact reducing vacuum bag according to claim 18, wherein re-entry of air is blocked in the first configuration.

20. The susceptibility artifact reducing vacuum bag according to any of claims 18-19, wherein the valve has a third configuration in which said valve is completed locked for air flowing in and out to/from the susceptibility artifact reducing vacuum bag.

21. The susceptibility artifact reducing vacuum bag according to any of the preceding claims, wherein the susceptibility artifact reducing vacuum bag is configured to conform to the neck of a human body.

22. The susceptibility artifact reducing vacuum bag according to any of the preceding claims, wherein the susceptibility artifact reducing vacuum bag is configured to conform to the foot, or leg, or knee, or arm, or hand, or shoulder, or back, or head of a human body.

23. The susceptibility artifact reducing vacuum bag according to any of the preceding claims, the susceptibility artifact reducing vacuum bag being substantially rectangular having a width less than 400 mm, or less than 350 mm, or less than 300 mm, or less than 250 mm, or less than 200 mm, or less than 150 mm, or less than 100 mm.

24. The susceptibility artifact reducing vacuum bag according to any of the preceding claims, the susceptibility artifact reducing vacuum bag being substantially rectangular having a length less than 1000 mm, or less than 900 mm, or less than 800 mm, or less than 700 mm, or less than 600 mm, or less than 500 mm, or less than 400 mm.

25. The susceptibility artifact reducing vacuum bag according to any of the preceding claims, wherein the susceptibility artifact reducing vacuum bag is configured to conform to at least one breast of a human body.

26. The susceptibility artifact reducing vacuum bag according to any of the preceding claims, the susceptibility artifact reducing vacuum bag being substantially cup-shaped or substantially conical or substantially hemispherical.

27. The susceptibility artifact reducing vacuum bag according to any of the preceding claims, configured to be placed in recess, preferably a cushioned recess, wherein a breast is positioned in the recess.

28. The susceptibility artifact reducing vacuum bag according to claim 27, configured such that the breast hangs freely in relation to the recess, the susceptibility artifact reducing vacuum bag being configured to conform tightly to the breast.

29. The susceptibility artifact reducing vacuum bag according to any of the preceding claims, wherein the diamagnetic material is pyrolytic graphite.

30. The susceptibility artifact reducing vacuum bag according to any of the preceding claims, wherein the filler material is a polymer, such as a synthetic polymer selected from the group of thermoplastics, thermosets, elastomers and synthetic fibers.

31. The susceptibility artifact reducing vacuum bag according to any of the preceding claims, wherein the mixture comprises from about 4% v/v to about 12% v/v of the diamagnetic material, or about 5% v/v to about 11% v/v of the diamagnetic material, or about 6% v/v to about 10% v/v of the diamagnetic material, or about 7% v/v to about 9% v/v of the diamagnetic material, or about 7.5% v/v to about 8.5% v/v of the diamagnetic material, or about 7.8% v/v to about 8.2% v/v of the diamagnetic material, or about 7.9% v/v to about 8.1% v/v of the diamagnetic material, such as about 8.0% v/v, or about 8.1% v/v, or about 8.2% v/v, or about 8.3% v/v, or about 8.4% v/v, or about 8.5% v/v, or about 7.9% v/v, or about 7.8% v/v, or about 7.7% v/v, or about 7.6% v/v, or about 7.5% v/v of the diamagnetic material.

32. A method for reducing local magnetic inhomogeneity of a portion of a body inside a magnetic resonance imaging system, the method comprising the steps of:
providing a susceptibility artifact reducing vacuum bag comprising a mixture of diamagnetic composite material made of a diamagnetic material and a filler material, the fraction of diamagnetic composite material and filler material selected such that the mixture has a net magnetic susceptibility corresponding substantially to the magnetic susceptibility of human tissue;
positioning the bag on and/or around the portion of the body, wherein the vacuum bag is in a non-vacuum configuration;
establishing a vacuum in the vacuum bag, thereby entering a vacuum configuration of the vacuum bag, wherein the vacuum bag conforms tightly to the body.

33. The method according to claim 32, wherein the vacuum bag is loose and flexible in the non-vacuum configuration.

34. The method according to any of claims 32-33, wherein the vacuum bag is substantially stiff in the vacuum configuration.

35. The method according to any of claims 32-34, wherein the bag is positioned around the neck of a wearer.

36. The method according to any of claims 32-35, using the vacuum bag of any of claims 1-31.
